(19) United States
(12) Patent Application Publication
(10) Pub. No.: US 2004/0209254 A1
(43) Pub. Date: Oct. 21, 2004

(54) Diagnostic polymorphisms for the tgf-beta1 promoter

(76) Inventor: Lee A. Henderson, Ithaca, NY (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/333,892

(22) PCT Filed: Jul. 25, 2001
(86) PCT No.: PCT/US01/23368

Related U.S. Application Data
(60) Provisional application No. 60/220,583, filed on Jul. 25, 2000.

(51) Int. Cl. C12Q 1/68

(52) U.S. Cl. 435/6

(57) ABSTRACT

Disclosed are single nucleotide polymorphisms (SNPs) associated with breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecysteclomy degenerative joint disease, end stage renal disease and frequent de-clots, end stage renal disease due to focal segmental glomerular sclerosis, end stage renal disease due to insulin dependent diabetes mellitus, and seizure disorder. Also disclosed are methods for using the SNPs to determine susceptibility to these diseases; nucleotide sequences containing the SNPs; kits for determining the presence of the SNPs; and methods of treatment or prophylaxis based on the presence of the SNPs.

### BACKGROUND

This invention relates to detection of individuals at risk for pathological conditions based on the presence of single nucleotide polymorphisms (SNPs) at positions 216 and 563 on the TGF-β1 Promoter.

During the course of evolution, spontaneous mutations appear in the genomes of organisms. It has been estimated that variations in genomic DNA sequences are created continuously at a rate of about 100 new single base changes per individual (Kondrashow, J. Theor. Biol., 175:583-594, 1995; Crow, Exp. Clin. Immunogenet., 12:121-128, 1995). These changes in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome are single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives (alleles) exist in a population. In addition, the least frequent allele must occur at a frequency of 1% or greater. Several definitions of SNPs exist in the literature (Brooks, Gene, 234:177-186, 1999). As used herein, the tend “single nucleotide polymorphism” or “SNP” includes all single base variants and so includes nucleotide insertions and deletions in addition to single nucleotide substitutions (e.g. A->G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998). The frequency of SNPs varies with the type and location of the change. In base substitutions, two-thirds of the substitutions involve the C<->T (G<->A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct, as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect, where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP)(U.S. Pat. Nos. 5,324,631; 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases has also been used to detect SNPs.

There is growing recognition that SNPs can provide a powerful tool for the detection of individuals whose genetic make-up alters their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

### SUMMARY

The present inventor has discovered novel single nucleotide polymorphisms (SNPs) associated with the development of various diseases including breast cancer, prostate cancer stage D, colon cancer, lung cancer, hypertension (HTN), atherosclerotic peripheral vascular disease due to hypertension (ASPVD due to HTN), cerebrovascular accident due to hypertension (CVA due to HTN), cataracts due to hypertension (CAT due to HTN), hypertensive cardiomyopathy (HTN CM), myocardial infarction due to hypertension (MI due to HTN), end stage renal disease due to hypertension (ESRD due to HTN), non-insulin dependent diabetes mellitus (NIDDM), atherosclerotic peripheral vascular disease due to non-insulin dependent diabetes mellitus (ASPVD due to NIDDM), cerebrovascular accident due to non-insulin dependent diabetes mellitus (CVA dule to NIDDM), ischemic cardiomyopathy (ischemic CM), ischemic cardiomyopathy with non-insulin dependent diabetes mellitus (ischemic CM with NIDDM), myocardial infarction due to non-insulin dependent diabetes mellitus (MI due to NIDDM), atrial fibrillation without valvular disease (afib without valvular disease), alcohol abuse, anxiety, asthma, chronic obstructive pulmonary disease (COPD), cholecystectomy, degenerative joint disease (DJD), end stage renal disease and frequent de-clots (ESRD and frequent de-clots), end stage renal disease due to focal segmental glomerular sclerosis (ESRD due to FSGS), end stage renal disease due to insulin dependent diabetes mellitus (ESRD due to IDDM), and seizure disorder. As such, these polymorphisms provide a method for diagnosing a genetic predisposition for the development of these diseases in individuals. Information obtained from the detection of SNPs associated with the development of these diseases is of great value in their treatment and prevention.

Accordingly, one aspect of the present invention provides a method for diagnosing a genetic predisposition for breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischernic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder in a subject, comprising obtaining a sample containing at least one polynucleotide from the subject, and analyzing the polynucleotide to detect a genetic polymorphism wherein said genetic polymorphism is associated with an altered susceptibility to developing breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the polymorphism is located in the TGF-β1 gene.

Another aspect of the present invention provides an isolated nucleic acid sequence comprising at least 10 contiguous nucleotides from SEQ ID NO: 1, or their complements, wherein the sequence contains at least one polymorphic site associated with a disease and in particular breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention is a kit for the detection of a polymorphism comprising, at a minimum, at least one polynucleotide of at least 10 contiguous nucleotides of SEQ ID NO: 1, or their complements, wherein the polynucleotide contains at least one polymorphic site associated with breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder.

Yet another aspect of the invention provides a method for treating breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder comprising, obtaining a sample of biological material containing at least one polynucleotide from the subject; analyzing the polynucleotide to detect the presence of at least one polymorphism associated with breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder; and treating the subject in such a way as to counteract the effect of any such polymorphism detected.

Still another aspect of the invention provides a method for the prophylactic treatment of a subject with a genetic predisposition to breast cancer, prostate cancer stage D, colon cancer, lung cancer, HTN, ASPVD due to HTN, CVA due to HTN, CAT due to HTN, HTN CM, MI due to HTN, ESRD due to HTN NIDDM, ASPVD due to NIDDM, CVA due to NIDDM, ischelmic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder comprising, obtaining a sample of biological material containing at least one polyiiucleotide from the subject; analyzing the polynucleotide to detect the presence of at least one polymorphism associated with breast cancer, prostate cancer stage D, colon cancer, lung cancer, HTN, ASPVD due to HTN, CVA due to HTN, CAT due to HTN, HTN CM, MI due to HTN, ESRD due to HTN, NEDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder; and treating the subject.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### BRIEF DESCRIPTION OF THE DRAWINGS

These and other features, aspects, and advantages of the present invention will become better understood with regard to the following description, appended claims, and accompanying drawings where:

FIG. 1 shows SEQ ID NO: 1, the nucleotide sequence of the TGF-β1 promoter region as contained in GenBank (accession no. J04431). Thus, all nucleotides will be positively numbered, rather than bear negative numbers reflecting their position upstream from the transcription initiation site, a scheme often used for promoters. The two numbering systems can be interconverted, if necessary. According to the annotation of Accession Number J04431, there are two major transcription initiation sites (at positions +1363 and +1633), and two minor transcription initiation sites (at positions+1832 and +1887), so the choice of which transcription initiation site to serve as the reference is not altogether clear.

The first SNP mentioned below (C216→G) is located at position 216 according to the numbering scheme of GenBank Accession Number J04431. The 20 nucleotides surrounding the SNP are as follows: 5′-TTC CCC CTC T [C/G] TCT CCT TTC C-3′ (nucleotides 206-226 of SEQ ID NO: 1).

The second SNP mentioned below (G563→A) is located at position 563 according to the numbering scheme of GenBank Accession Number J04431. The 20 nucleotides surrounding the SNP are as follows: 5′-TGC CTC CAA C [G/A] TCA CCA CCA T-3′ (nucleotides 553-573 of SEQ ID NO: 1).

The sequence J04431 does not contain a translation initiation site.

### DEFINITIONS

nt=nucleotide

bp=base pair

kb=kilobase; 1000 base pairs

ASPVD=atherosclerotic peripheral vascular disease

COPD=chronic obstructive pulmonary disease

CVA=cerebrovascular accident

DJD=degenerative joint disease, also know as osteoarthritis

DOL=dye-labeled oligonucleotide ligation assay

ESRD=end-stage renal disease

FSGS=focal segmental glomerular sclerosis

HTN=hypertension

MASDA=multiplexed allele-specific diagnostic assay

MADGE=microtiter array diagonal gel electrophoresis

MI=myocardial infarction

NIDDM=noninsulin-dependent diabetes mellitus

OLA=oligonucleotide ligation assay

PCR=polymerase chain reaction

RFLP=restriction fragment length polymorphism

SNP=single nucleotide polymorphism

“Polynucleotide” and “oligonucleotide” are used interchangeably and mean a linear polymer of at least 2 nucleotides joined together by phosphodiester bonds and may consist of either ribonucleotides or deoxyribonucleotides.

“Sequence” means the linear order in which monomers occur in a polymer, for example, the order of amino acids in a polypeptide or the order of nucleotide in a polynucleotide.

“Polymorphism” refers to a set of genetic variants at a particular genetic locus among individuals in a population.

“Promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns”) between individual coding segments (“exons”). A promoter is herein considered as a part of the corresponding gene. Coding refers to the representation of amino acids, start and stop signals in a three base “triplet” code. Promoters are often upstream (“5′ to”) the transcription initiation site of the gene.

“Gene therapy” means the introduction of a functional gene or genes from some source by any suitable method into a living cell to correct for a genetic defect.

“Wild type allele” means the most frequently encountered allele of a given nucleotide sequence of an organism.

“Genetic variant” or “variant” means a specific genetic variant which is present at a particular genetic locus in at least one individual in a population and that differs from the wild type.

As used herein the terms “patient” and “subject” are not limited to human beings, but are intended to include all vertebrate animals in addition to human beings.

As used herein the terms “genetic predisposition”, “genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (wild type) by at least 1%, preferably by at least 2%, more preferably by at least 4% and more preferably still by at least 8%. Alternatively, an odds ratio of 1.5 was chosen as the threshold of significance based on the recommendation of Austin et al. in Epidemiol. Rev., 16:65-76, 1994. “[E]pidemiology in general and case-control studies in particular are not well suited for detecting weak associations (odds ratios<1.5).” Id. at 66.

As used herein “isolated nucleic acid” means a species of the invention that is the predominate species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

### TGF-β1 Signalling

Excess TGF-β1 signalling has been associated with growth inhibition and apoptosis, whereas decreased TGF-β1 signalling has been associated with cell proliferation. For example, numerous animal and human studies have linked the progression of renal disease, especially its hallmark pathology of interstitial fibrosis and glomerular sclerosis, to increased signalling by TGF-β1. Signalling by TGF-β1 involves specific binding of the ligand to the type II TGF-β1 receptor (abbreviated as TGFβ-RII), present on the plasma membrane of target cells such as fibroblasts in the case of glomerular and interstitial fibrosis. This receptor-ligand complex then heterodimerizes with the type I TGF-β1 receptor (abbreviated as TGFβ-RI). TGFβ-RI is constitutively active. Like the concentrations of ligand (TGF-β1) and TGFβ-RI, the concentration of TGFβ-RII in the plasma membrane are likely to be rate-limiting for signalling by TGF-β1. All elements of the pathway appear to be subject to complex regulation.

If the level of TGFβ-RII gene product (i.e. protein) is proportional to the level of mRNA, and the mRNA level is proportional to the transcriptional rate of the gene, then a SNP which disrupts a transcriptional activator site would be expected to decrease both the rate of transcription of the gene and the eventual concentration of TGFβ-RII in the plasma membrane of cells which express this protein. The net effect of such a SNP is expected to be protection against renal failure.

TGF-β1 also inhibits cellular proliferation in a number of cell types. Signalling by TGF-β1 is thus expected to be depressed in individuals with a predisposition to malignancies.

### Novel Polymorphisms

The present application provides single nucleotide polymorphisms (SNPs) in a gene associated of breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. The polymorphisms are a C to G transversion found in the TGF-β1 promoter at position 216 and a G to A transition found in the TGF-β1 promoter at position 563.

### Preparation of Samples

The presence of genetic variants in the above genes or their control regions, or in any other genes that may affect susceptibility to disease is determined by screening nucleic acid sequences from a population of individuals for such variants. The population is preferably comprised of some individuals with the disease of interest, so that any genetic variants that are found can be correlated with disease. The population is also preferably comprised of some individuals that have known risk for the disease. The population should preferably be large enough to have a reasonable chance of finding individuals with the sought-after genetic variant. As the size of the population increases, the ability to find significant correlations between a particular genetic variant and susceptibility to disease also increases.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad. Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

### Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In addition, the allele frequency of the genetic variant in a population or subpopulation with the disease or pathology should vary from its allele frequency in the population without the disease or pathology (control population) or the control sequence (wild type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

RFLP analysis (see, e.g. U.S. Pat. No. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6. ). Double strands are first heat-denatured. The single strands are then subjected to polyacrylamide gel electrophoresis under non-denaturing conditions at constant temperature (i.e., low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al., Trends Genet. 7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine (T) when mismatched with cytosine, guanine or thymine and the chemical reactivity of cytosine (C) when mismatched with thymine, adenine or cytosine (Cotton et al., Proc. Natl. Acad. Sci. USA, 85:4397-4401, 1988). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998 and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Aiolec. Genet., 6:337-347, 1997). In MASDA, samples from multiplex PCR are immobilized on a solid support. A single hybridization is conducted with a pool of labeled allele specific oligonucleotides (ASO). Any ASOs that hybridize to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end, such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE) (Day and Humphries, Anal. Biochem., 222:389-395, 1994). In this assay restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sommer et al., Mayo Clin. Proc., 64:1361-13721989; Sarker et al., Anal. Biochem. 1990), allele-specific amplification (ASA), allele-specific PCR, and amplification refractory mutation system (ARMS) (Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determined whether an individual is homozygous or heterozygous for the mutation (Dutton and Sommer, BioTechniques, 11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers has a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can be used as a template.

In one method for mass screening for SNPs based on the OLA, amplified DNA templates are analyzed for their ability to serve as templates for ligation reactions between labeled oligonucleotide probes (Samotiali et al., Genomics, 20:238-242, 1994). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thermostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J. Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumakler et al., Human Mut., 7:346-354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled dNTP and noncompeting ddNTPs. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are not suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an increased risk of developing a disease, condition or disorder, and in particular breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the genetic variant is at one of the polymorphic sites contained in Table 11. In another embodiment, the genetic variant is one of the variants contained in Table 11 or the complement of any of the variants contained in Table 11. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1, or the complements of SEQ ID NO 1. In one preferred embodiment, the sequence contains at least one polymiorphic site associated with a disease, and in particular breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. In one embodiment, the genetic variant is at one of the polymorphic sites contained in Table 11. In another embodiment, the genetic variant is one of the variants contained in Table 11 or the complement of any of the variants contained in Table 11. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder. The kits contain, at a minimum, at least one polynucleotide of at least 10 contiguous nucleotides of SEQ ID NO 1, or the complements of SEQ ID NO: 1. In one embodiment, the genetic variant is at one of the polymorphic sites contained in Table 11. Alternatively the 3′ end of the polynucleotide is immediately 5′ to a polymorphic site, preferably a polymorphic site selected from the sites in Table 11. In another embodiment, the genetic variant is one of the variants contained in Table 11 or the complement of any of the variants contained in Table 11. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder and in particular breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder caused either directly or indirectly by the presence of one or more single nucleotide polymorphisms. In this method genetic material from a patient, for example, DNA, cDNA, RNA or mRNA is screened for the presence of one or more SNPs associated with the disease of interest. Depending on the type and location of the SNP, a treatment regime is designed to counteract the effect of the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder, and in particular breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischemic CM with NIDDM, MI due to NIDDM, afib without valvular disease, alcohol abuse, anxiety, asthma, COPD, cholecystectomy, DJD, ESRD and frequent de-clots, ESRD due to FSGS, ESRD due to IDDM, or seizure disorder due to the presence of one or more single nucleotide polymorphisms. In this embodiment, genetic material, such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

### EXAMPLES

Positions of the single nucleotide polymorphisms (SNP) are given according to the numbering scheme in GenBank Accession Number J04431. Thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://www.ncbi.nlim.nih.gov/

In the following examples, SNPs are written as “reference sequence” (or “wild type”) nucleotide” →“variant nucleotide.” Changes in nucleotide sequences are indicated in bold print. The standard nucleotide abbreviations are used in which A=adenine, C=cytosine, G=guanine, T=thymine, M=A or C, R=A or G, W=A or T, S=C or G, Y=C or T,K=G or T, V=A or C or G, H=A or C or T; D=A or G or T; B=C or G or T; N=A or C or G or T.

### Example 1

### Detection of Novel Polymorphisms by Direct Sequencing of Leukocyte Genomic DNA

Leukocytes were obtained from human whole blood collected with EDTA as an anticoagulant. Blood was obtained from a group of African-American men, African-American women, Caucasian men, and Caucasian women without any known disease. Blood was also obtained from individuals with breast cancer, prostate cancer stage D, colon cancer, lung cancer, HTN, ASPVD due to HTN, CVA due to HTN, CAT due to HTN, HTN CM, MI due to HTN, ESRD due to HTN, NIDDM, ASPVD due to NIDDM, CVA due to NIDDM, ischemic CM, ischeniic CM with NIDDM, MI due to NIDDM, afib without valvuilar disease, alcohol abuse, anxiety, asthma, COPD, cholecystectolniy, DJD, ESRD and frequent de-clots, ESRD due to FSGS, ESRD due to IDDM, or seizure disorder as indicated in the tables below.

Genomic DNA was purified from the collected leukocytes using standard protocols well known to those of ordinary skill in the art of molecular biology (Ausubel et al., Short Protocol in Molecular Biology, 3^{rd} ed., John Wiley and Sons, 1995; Sambrook et al., Molecular Cloning, Cold Spring Harbor Laboratory Press, 1989; and Davis et al., Basic Methods in Molecular Biology, Elsevier Science Publishing, 1986). One hundred nanograms of purified genomic DNA were used in each PCR reaction.

Standard PCR reaction conditions were used. Methods for conducting PCR are well known in the art and can be found, for example, in U.S. Pat. Nos 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990. Two sets of primers were used. The sense primer for the C216→GSNP was 5′-CCT TTC CCC TCT CTC TCC TTT -3′ (SEQ ID NO: 2). The anti-sense primer was 5′-GAT GGT GGT GAC GTT GGA G-3′ (SEQ ID NO: 3). The PCR product produced spanned positions 66 to 265 of the human TGF-β1 gene (SEQ ID NO: 1). The sense primer for the G563→A SNP was 5′-TGC ATG GGG ACA CCA TCT ACA G-3′(SEQ ID NO: 4). The antisense primer was 5′ TCT TGA CCA CTG TGC CAT CCT C-3′ (SEQ ID NO: 5). The PCR product spanned positions 421-622 of the human TGP-l1 gene (SEQ ID NO: 1).

Twenty-five ng of template leukocyte genomic DNA was used for each PCR amplification. Twenty-five microliters of an aqueous solution of genomic DNA (1 ng/ul) was dispensed to the wells of a 96-well plate, and dried down at 70° C. for 15 min. The DNA was rehydrated with 7 ul of ultra-pure but not autoclaved water (Milli-Q, Millipore Corp.). PCR conditions were as follows: 5 min. at 94° C., followed by 35 cycles, where each cycle consisted of 45 seconds at 94° C. to denature the double-stranded DNA, then 45 seconds at 65° C. for specific annealing of primers to the single-stranded DNA, followed by 45 seconds at 72° C. for extension. After the 35th cycle, the reaction mixture was held at 72° C. for 10 min. for a final extension reaction.

The PCR reaction contained a total volume of 20 microliters (ul), and consisted of 10 ul of a premade PCR reaction mix (Sigma “JumpStart Ready Mix with RED Taq Polymerase”). Primers at 10 uM were diluted to a final concentration of 0.3 uM in the PCR reaction mix. Post-PCR clean-up was performed prior to submission of PCR product to sequencing.

Pyrosequencing is a method of sequencing DNA by synthesis, where the addition of one of the four dNTPs that correctly matches the complementary base on the template strand is detected. Detection occurs via utilization of the pyrophosphate molecules liberated upon base addition to the elongating synthetic strand. The pyrophosphate molecules are used to make ATP, which in turn drives the emission of photons in a luciferin/luciferase reaction, and these photons are detected by the instrument. A Luc96 Pyrosequencer was used under default operating condition supplied by the manufacturer. Primers were designed to anneal within 5 bases of the polymorphism, to serve as sequencing primers. Patient genomic DNA was subject to PCR using amplifying primers that amplify an approximately 200 base pair amplicon containing the polymorphisms of interest. One the amplifying primers, whose orientation is opposite to the sequencing primer, was biotinylated. This allowed selection of single stranded template for pyrosequencing, whose orientation is complementary to the sequencing primer. Amplicons prepared from genomic DNA were isolated by binding them to streptavidin-coated magnetic beads. After denaturation in NaOH, the biotinylated strands were separated from their complementary strands using magnetics.

After washing the magnetic beads, the biotinylated template strands still bound to the beads were transferred into 96-well plates. The sequencing primers were added, annealing was carried out at 95° C. for 2 minutes, and plates were placed in the Pyrosequencer. The enzymes, substrates and dNTPs used for synthesis and pyrophosphate detection were added to the instrument immediately prior to sequencing.

The Luc96 software requires definition of a program of adding the four dNTPs that is specific for the location of the sequencing primer, the DNA composition flanking the SNP, and the two possible alleles at the polymorphic locus. This order of adding the bases generates theoretical outcomes of light intensity patterns for each of the two possible homozygous states and the single heterozygous state. The Luc96 software then compares the actual outcome to the theoretical outcome and calls a genotype for each well. Each sample is also assigned one of three confidence scores: pass, uncertain, fail. The results for each plate are output as a text file and processed in Excel using a Visual Basic program to generate a report of genotype and allele frequencies for the various disease and population cell groupings represented on the 96 well plate.

Prediction of potential transcription binding factor sites was performed using a commercially available software program [GENOMATIX MatInspector Professional release 4.2, February, 2000; URL: http://genomatix.gsf.de/cgibin/matinspector/matinspector.p1; (Quandt K et al., Nucleic Acids Res., 23: 4878-4884 (1995)].

### Example 2

### C to G Transversion at Position 216 of Human TGF-β1 Promoter

TABLE 1
ALLELE FREQUENCY

| Disease | Race | CHROMOSOMES | N | C | N | G |
|---|---|---|---|---|---|---|
| Controls | African-American | 88 | 87 | 98.9% | 1 | 1.1% |
| | Caucasian | 92 | 92 | 100.0% | 0 | 0.0% |
| Breast Cancer | African-American | 24 | 23 | 95.8% | 1 | 4.2% |
| | Caucasian | 22 | 22 | 100.0% | 0 | 0.0% |
| Prostate cancer stage D | African-American | 24 | 23 | 95.8% | 1 | 4.2% |
| | Caucasian | 24 | 24 | 100.0% | 0 | 0.0% |
| Colon cancer | African-American | 46 | 46 | 100.0% | 0 | 0.0% |
| | Caucasian | 44 | 43 | 97.7% | 1 | 2.3% |
| Hypertension | African-American | 44 | 43 | 97.7% | 1 | 2.3% |
| | Caucasian | 44 | 44 | 100.0% | 0 | 0.0% |
| ASPVD due to HTN | African-American | 54 | 52 | 96.3% | 2 | 3.7% |
| | Caucasian | 50 | 50 | 100.0% | 0 | 0.0% |
| CVA due to HTN | African-American | 44 | 44 | 100.0% | 0 | 0.0% |
| Cataracts due to HTN | African-American | 48 | 44 | 91.7% | 4 | 8.3% |
| | Caucasian | 44 | 42 | 95.5% | 2 | 4.5% |
| HTN CM | African-American | 48 | 46 | 95.8% | 2 | 4.2% |
| | Caucasian | 46 | 46 | 100.0% | 0 | 0.0% |
| MI due to HTN | African-American | 42 | 41 | 97.6% | 1 | 2.4% |
| | Caucasian | 46 | 46 | 100.0% | 0 | 0.0% |
| ESRD due to HTN | African-American | 44 | 42 | 95.5% | 2 | 4.5% |
| | Caucasian | 46 | 46 | 100.0% | 0 | 0.0% |
| NIDDM | African-American | 48 | 47 | 97.9% | 1 | 2.1% |
| | Caucasian | 48 | 48 | 100.0% | 0 | 0.0% |
| ASPVD due to NIDDM | African-American | 46 | 45 | 97.8% | 1 | 2.2% |
| | Caucasian | 48 | 48 | 100.0% | 0 | 0.0% |
| CVA due to NIDDM | African-American | 48 | 46 | 95.8% | 2 | 4.2% |
| | Caucasian | 46 | 46 | 100.0% | 0 | 0.0% |
| Ischemic CM | African-American | 48 | 45 | 93.8% | 3 | 6.3% |
| | Caucasian | 42 | 42 | 100.0% | 0 | 0.0% |
| Ischemic CM with NIDDM | African-American | 46 | 44 | 95.7% | 2 | 4.3% |
| | Caucasian | 46 | 46 | 100.0% | 0 | 0.0% |
| MI due to NIDDM | African-American | 48 | 47 | 97.9% | 1 | 2.1% |
| | Caucasian | 48 | 48 | 100.0% | 0 | 0.0% |
| Afib without valvular disease | African-American | 48 | 45 | 93.8% | 3 | 6.3% |
| | Caucasian | 48 | 48 | 100.0% | 0 | 0.0% |
| Alcohol abuse | African-American | 48 | 46 | 95.8% | 2 | 4.2% |
| | Caucasian | 48 | 48 | 100.0% | 0 | 0.0% |
| Anxiety | African-American | 48 | 44 | 91.7% | 4 | 8.3% |
| | Caucasian | 42 | 41 | 97.6% | 1 | 2.4% |
| Asthma | African-American | 48 | 44 | 91.7% | 4 | 8.3% |
| | Caucasian | 48 | 48 | 100.0% | 0 | 0.0% |
| COPD | African-American | 40 | 37 | 92.5% | 3 | 7.5% |
| Cholecystectomy | African-American | 48 | 47 | 97.9% | 1 | 2.1% |
| | Caucasian | 48 | 48 | 100.0% | 0 | 0.0% |
| DJD | African-American | 40 | 37 | 92.5% | 3 | 7.5% |
| | Caucasian | 40 | 40 | 100.0% | 0 | 0.0% |
| ESRD and frequent de-clots | African-American | 48 | 44 | 91.7% | 4 | 8.3% |
| | Caucasian | 42 | 42 | 100.0% | 0 | 0.0% |
| ESRD due to FSGS | African-American | 42 | 41 | 97.6% | 1 | 2.4% |
| | Caucasian | 44 | 44 | 100.0% | 0 | 0.0% |
| ESRD due to IDDM | African-American | 48 | 46 | 95.8% | 2 | 4.2% |
| | Caucasian | 48 | 47 | 97.9% | 1 | 2.1% |
| Seizure disorder | African-American | 46 | 43 | 93.5% | 3 | 6.5% |
| | Caucasian | 46 | 46 | 100.0% | 0 | 0.0% |

Additionally, it is necessary to disclose the make-up of the control groups by gender for purposes of calculating the data for men with prostate cancer. All other data was calculated without respect to gender. The allele frequency gender data for the control group is given in Table 2.

TABLE 2
ALLELE FREQUENCY GENDER DATA FOR
CONTROL GROUP

| | | CHROMO- | | | | |
|---|---|---|---|---|---|---|
| Disease | Race | SOMES | N | C | N | G |
| Controls | Black men | 46 | 45 | 97.8% | 1 | 2.2% |
| | Black women | 42 | 42 | 100.0% | 0 | 0.0% |
| | White men | 44 | 44 | 100.0% | 0 | 0.0% |
| | White women | 48 | 48 | 100.0% | 0 | 0.0% |

TABLE 3
GENOTYPE FREQUENCY

| Disease | Race | People | N | C/C | N | C/G | N | G/G |
|---|---|---|---|---|---|---|---|---|
| Controls | African-American | 44 | 43 | 97.7% | 1 | 2.3% | 0 | 0.0% |
| | Caucasian | 46 | 46 | 100.0% | 0 | 0.0% | 0 | 0.0% |
| Breast cancer | African-American | 12 | 11 | 91.7% | 1 | 8.3% | 0 | 0.0% |
| | Caucasian | 11 | 11 | 100.0% | 0 | 0.0% | 0 | 0.0% |
| Prostate cancer stage D | African-American | 12 | 11 | 91.7% | 1 | 8.3% | 0 | 0.0% |
| | Caucasian | 12 | 12 | 100.0% | 0 | 0.0% | 0 | 0.0% |
| Colon cancer | African-American | 23 | 23 | 100.0% | 0 | 0.0% | 0 | 0.0% |
| | Caucasian | 22 | 21 | 95.5% | 1 | 4.5% | 0 | 0.0% |
| Hypertension | African-American | 22 | 21 | 95.5% | 1 | 4.5% | 0 | 0.0% |
| | Caucasian | 22 | 22 | 100.0% | 0 | 0.0% | 0 | 0.0% |
| ASPVD due to HTN | African-American | 27 | 25 | 92.6% | 2 | 7.4% | 0 | 0.0% |
| | Caucasian | 25 | 25 | 100.0% | 0 | 0.0% | 0 | 0.0% |
| CVA due to HTN | African-American | 22 | 22 | 100.0% | 0 | 0.0% | 0 | 0.0% |
| Cataracts due to HTN | African-American | 24 | 20 | 83.3% | 4 | 16.7% | 0 | 0.0% |
| | Caucasian | 22 | 20 | 90.9% | 2 | 9.1% | 0 | 0.0% |
| HTN CM | African-American | 24 | 22 | 91.7% | 2 | 8.3% | 0 | 0.0% |
| | Caucasian | 23 | 23 | 100.0% | 0 | 0.0% | 0 | 0.0% |
| MI due to HTN | African-American | 21 | 20 | 95.2% | 1 | 4.8% | 0 | 0.0% |
| | Caucasian | 23 | 23 | 100.0% | 0 | 0.0% | 0 | 0.0% |
| ESRD due to HTN | African-American | 22 | 20 | 90.9% | 2 | 9.1% | 0 | 0.0% |
| | Caucasian | 23 | 23 | 100.0% | 0 | 0.0% | 0 | 0.0% |
| NIDDM | African-American | 24 | 23 | 95.8% | 1 | 4.2% | 0 | 0.0% |
| | Caucasian | 24 | 24 | 100.0% | 0 | 0.0% | 0 | 0.0% |
| ASPVD due to NIDDM | African-American | 23 | 22 | 95.7% | 1 | 4.3% | 0 | 0.0% |
| | Caucasian | 24 | 24 | 100.0% | 0 | 0.0% | 0 | 0.0% |
| CVA due to NIDDM | African-American | 24 | 22 | 91.7% | 2 | 8.3% | 0 | 0.0% |
| | Caucasian | 23 | 23 | 100.0% | 0 | 0.0% | 0 | 0.0% |
| Ischemic CM | African-American | 24 | 21 | 87.5% | 3 | 12.5% | 0 | 0.0% |
| | Caucasian | 21 | 21 | 100.0% | 0 | 0.0% | 0 | 0.0% |
| Ischemic CM with NIDDM | African-American | 23 | 21 | 91.3% | 2 | 8.7% | 0 | 0.0% |
| | Caucasian | 23 | 23 | 100.0% | 0 | 0.0% | 0 | 0.0% |
| MI due to NIDDM | African-American | 24 | 23 | 95.8% | 1 | 4.2% | 0 | 0.0% |
| | Caucasian | 24 | 24 | 100.0% | 0 | 0.0% | 0 | 0.0% |
| Afib without valvular disease | African-American | 24 | 21 | 87.5% | 3 | 12.5% | 0 | 0.0% |
| | Caucasian | 24 | 24 | 100.0% | 0 | 0.0% | 0 | 0.0% |
| Alcohol abuse | African-American | 24 | 22 | 91.7% | 2 | 8.3% | 0 | 0.0% |
| | Caucasian | 24 | 24 | 100.0% | 0 | 0.0% | 0 | 0.0% |
| Anxiety | African-American | 24 | 20 | 83.3% | 4 | 16.7% | 0 | 0.0% |
| | Caucasian | 21 | 20 | 95.2% | 1 | 4.8% | 0 | 0.0% |
| Asthma | African-American | 24 | 20 | 83.3% | 4 | 16.7% | 0 | 0.0% |
| | Caucasian | 24 | 24 | 100.0% | 0 | 0.0% | 0 | 0.0% |
| COPD | African-American | 20 | 17 | 85.0% | 3 | 15.0% | 0 | 0.0% |
| Cholecystectomy | African-American | 24 | 23 | 95.8% | 1 | 4.2% | 0 | 0.0% |
| | Caucasian | 24 | 24 | 100.0% | 0 | 0.0% | 0 | 0.0% |
| DJD | African-American | 20 | 17 | 85.0% | 3 | 15.0% | 0 | 0.0% |
| | Caucasian | 20 | 20 | 100.0% | 0 | 0.0% | 0 | 0.0% |
| ESRD and frequent de-clots | African-American | 24 | 21 | 87.5% | 2 | 8.3% | 1 | 4.2% |
| | Caucasian | 21 | 21 | 100.0% | 0 | 0.0% | 0 | 0.0% |
| ESRD due to FSGS | African-American | 21 | 20 | 95.2% | 1 | 4.8% | 0 | 0.0% |
| | Caucasian | 22 | 22 | 100.0% | 0 | 0.0% | 0 | 0.0% |
| ESRD due to IDDM | African-American | 24 | 22 | 91.7% | 2 | 8.3% | 0 | 0.0% |
| | Caucasian | 24 | 23 | 95.8% | 1 | 4.2% | 0 | 0.0% |
| Seizure disorder | African-American | 23 | 20 | 87.0% | 3 | 13.0% | 0 | 0.0% |
| | Caucasian | 23 | 23 | 100.0% | 0 | 0.0% | 0 | 0.0% |

TABLE 4
GENOTYPE FREQUENCY GENDER DATA
FOR CONTROL GROUP

| Disease | Race | People | N | C/C | N | C/G | N | G/G |
|---|---|---|---|---|---|---|---|---|
| Controls | Black men | 23 | 22 | 95.7% | 1 | 4.4% | 0 | 0.0% |
| | Black | 21 | 21 | 100.0% | 0 | 0.0% | 0 | 0.0% |
| | women | | | | | | | |
| | White men | 22 | 22 | 100.0% | 0 | 0.0% | 0 | 0.0% |
| | White | 24 | 24 | 100.0% | 0 | 0.0% | 0 | 0.0% |
| | women | | | | | | | |

Allele-Specific Odds Ratios

The susceptibility or risk allele is indicated below, as well as the odds ratio (OR). Haldane's correction was used if the denominator is zero, and so indicated (“H”). If the odds ratio (OR) is ≧1.5, the 95% confidence interval (C.I.) is also given. An odds ratio of 1.5 is chosen as the threshold of significance based on the recommendation of Austin et al. In Epidemiol. Rev. 16:65-76, 1994.

“. . . [E]pidemiology in general and case-control studies in particular are not well suited for detecting weak associations (odds ratios <1.5).” Id. at 66.

An example of an odds ratio calculation is given below: Hypertension: African-Americans

| | Cases | Controls |
|---|---|---|
| G | 1 | 1 |
| C | 43 | 87 |

In this example, the odds ratio that the G allele is the susceptibility allele for African-Americans with hypertension is (1)(87)/(43)(1)=2.0. Odds ratios of 1.5 or greater are highlighted below.

TABLE 5
ALLELE-SPECIFIC ODDS RATIOS

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African-American | C | 1.6 | 0.1 | 39.9 | H |
| | Caucasian | G | 6.4 | 0.3 | 159.8 | H |
| Breast cancer | African-American | G | 3.8 | 0.2 | 62.8 | |
| | Caucasian | C | 1.0 | — | — | |
| Prostate cancer stage D* | African-American | G | 2.0 | 0.1 | 32.7 | |
| | Caucasian | C | 1.0 | — | — | |
| Hypertension | African-American | G | 2.0 | 0.1 | 33.1 | |
| | Caucasian | C | 1.0 | — | — | |
| ASPVD due to HTN*¹ | African-American | G | 1.7 | 0.1 | 18.9 | |
| | Caucasian | C | 1.0 | — | — | |
| CVA due to HTN*¹ | African-American | C | 3.1 | 0.1 | 77.4 | H |
| Cataracts due to HTN*¹ | African-American | G | 7.9 | 0.9 | 72.9 | |
| | Caucasian | G | 10.9 | 0.5 | 231.6 | H |
| ESRD due to HTN*¹ | African-American | G | 2.0 | 0.2 | 23.4 | |
| | Caucasian | C | 1.0 | — | — | |
| NIDDM | African-American | G | 1.9 | 0.1 | 30.3 | |
| | Caucasian | C | 1.0 | — | — | |
| ASPVD due to NIDDM*² | African-American | G | 1.0 | 0.1 | 17.2 | |
| | Caucasian | C | 1.0 | — | — | |
| CVA due to NIDDM*² | African-American | G | 2.0 | 0.2 | 23.3 | |
| | Caucasian | C | 1.0 | — | — | |
| Afib without valvular disease | African-American | G | 5.8 | 0.6 | 57.4 | |
| | Caucasian | C | 1.0 | — | — | |
| Alcohol abuse | African-American | G | 3.8 | 0.3 | 42.8 | |
| | Caucasian | C | 1.0 | — | — | |
| Anxiety | African-American | G | 7.9 | 0.9 | 72.9 | |
| | Caucasian | G | 6.7 | 0.3 | 167.6 | H |
| Asthma | African-American | G | 7.9 | 0.9 | 72.9 | |
| | Caucasian | C | 1.0 | — | — | |
| COPD | African-American | G | 7.1 | 0.7 | 70.1 | |
| Cholecystectomy | African-American | G | 1.9 | 0.1 | 30.3 | |
| | Caucasian | C | 1.0 | — | — | |
| DJD | African-American | G | 7.1 | 0.7 | 70.1 | |
| | Caucasian | C | 1.0 | — | — | |
| ESRD and frequent de-clots | African-American | G | 7.9 | 0.9 | 72.9 | |
| | Caucasian | C | 1.0 | — | — | |
| ESRD due to FSGS | African-American | G | 2.1 | 0.1 | 34.8 | |
| | Caucasian | C | 1.0 | — | — | |
| ESRD due to IDDM | African-American | G | 3.8 | 0.3 | 42.8 | |
| | Caucasian | G | 5.8 | 0.2 | 146.2 | H |
| Seizure disorder | African-American | G | 6.1 | 0.6 | 60.1 | |
| | Caucasian | C | 1.0 | — | — | |

*Derived from the data for men only.
*¹Compared to HTN alone.
*²Compared to NIDDM alone.

Genotype-Specific Odds Ratios

The susceptibility allele (S) is indicated; the alternative allele at this locus is defined as the protective allele (P). Also presented is the odds ratio (OR) for the SS and SP genotypes; the odds ratio for the PP genotype is defined as 1, since it serves as the reference group, and is not presented separately. For odds ratios ≧1.5, the 95% confidence interval (C.I.) is also given in parentheses. An odds ratio of 1.5 was chosen as the threshold of significance based on the recommendation of Austin et al. in Epidemiol. Rev. 16:65-76, 1994. “[E]pidemiology in general and case-control studies in particular are not well suited for detecting weak associations (odds ratios<1.5).” Id. at 66.

An example is worked below, assuming that C is the susceptibility allele (S), and G is the protective allele (P).

Colon Cancer: African-American

| | Cases | Controls |
|---|---|---|
| CC (SS) | 23 | 43 |
| CG (SP) | 0 | 1 |
| GG (PP) | 0 | 0 |

Applying Haldatne's correction only where the denominator contains a 0, the above 2×3 table becomes:

Colon Cancer: African-American

| | Cases | Controls | Odds Ratio |
|---|---|---|---|
| CC (SS) | 47 | 87 | (47)(1)/(1)(87) = 0.5 |
| CG (SP) | 1 | 3 | (1)(1)/(3)(1) = 0.3 |
| GG (PP) | 1 | 1 | 1.0 (by definition) |

Odds ratios of 1.5 or higher are high-lighted below. Where Haldane's zero cell correction was used, the odds ratio is so indicated with an “H”.

TABLE 6
GENOTYPE-SPECIFIC ODDS RATIOS

| | | RISK | SS | | SP | |
|---|---|---|---|---|---|---|
| Disease | Race | ALLELE | O.R. | HALDANE | O.R. | HALDANE |
| Colon cancer | African-American | C | 0.5 | H | 0.3 | H |
| | Caucasian | G | 0.5 | H | 3.0 | H |
| Breast cancer | African-American | G | 0.3 | H | 1.0 | H |
| | Caucasian | C | 0.2 | H | 1.0 | H |
| Prostate cancer | African-American | G | 0.5 | H | 1.0 | H |
| stage D* | Caucasian | C | 0.6 | H | 1.0 | H |
| Hypertension | African-American | G | 0.5 | H | 1.0 | H |
| | Caucasian | C | 0.5 | H | 1.0 | H |
| ASPVD due to | African-American | G | 1.2 | H | 1.7 | H |
| HTN*¹ | Caucasian | C | 1.1 | H | 1.0 | H |
| CVA due to HTN*¹ | African-American | C | 1.0 | H | 0.3 | H |
| Cataracts due to | African-American | G | 0.5 | H | 3.0 | H |
| HTN*¹ | Caucasian | G | 0.4 | H | 5.0 | H |
| ESRD due to | African-American | G | 1.0 | H | 1.7 | H |
| HTN*¹ | Caucasian | C | 1.0 | H | 1.0 | H |
| NIDDM | African-American | G | 0.5 | H | 1.0 | H |
| | Caucasian | C | 0.5 | H | 1.0 | H |
| ASPVD due to | African-American | G | 1.0 | H | 1.0 | H |
| NIDDM*² | Caucasian | C | 1.0 | H | 1.0 | H |
| CVA due to | African-American | G | 1.0 | H | 1.7 | H |
| NIDDM*² | Caucasian | C | 1.0 | H | 1.0 | H |
| Afib without | African-American | G | 0.5 | H | 2.3 | H |
| valvular disease | Caucasian | C | 0.5 | H | 1.0 | H |
| Alcohol abuse | African-American | G | 0.5 | H | 1.7 | H |
| | Caucasian | C | 0.5 | H | 1.0 | H |
| Anxiety | African-American | G | 0.5 | H | 3.0 | H |
| | Caucasian | G | 0.4 | H | 3.0 | H |
| Asthma | African-American | G | 0.5 | H | 3.0 | H |
| | Caucasian | C | 0.5 | H | 1.0 | H |
| COPD | African-American | G | 0.4 | H | 2.3 | H |
| Cholecystectomy | African-American | G | 0.5 | H | 1.0 | H |
| | Caucasian | C | 0.5 | H | 1.0 | H |
| DJD | African-American | G | 0.4 | H | 2.3 | H |
| | Caucasian | C | 0.4 | H | 1.0 | H |
| ESRD and frequent | African-American | G | 0.0 | | 0.0 | |
| de-clots | Caucasian | C | 0.5 | H | 1.0 | H |
| ESRD due to FSGS | African-American | G | 0.5 | H | 1.0 | H |
| | Caucasian | C | 0.5 | H | 1.0 | H |
| ESRD due to | African-American | G | 0.5 | H | 1.7 | H |
| IDDM | Caucasian | G | 0.5 | H | 3.0 | H |
| Seizure disorder | African-American | G | 0.5 | H | 2.3 | H |
| | Caucasian | C | 0.5 | H | 1.0 | H |

*Derived from the data for men only.
*¹Compared to HTN alone.
*²Compared to NIDDM alone.

PCR and sequencing were conducted as described in Example 1. The primers used were those described in Example 1 for detection of the SNP at position 216. The control samples were in good agreement with Hardy-Weinberg equilibrium, as follows:

A frequency of 1.00 for the C allele (“q”) and 0 for the G allele (“p”) among Caucasian control individuals predicts genotype frequencies of 100% C/C, 0% C/G, and 0% G/G at Hardy-Weinberg equilibrium (p²+2pq+q²=1). The observed genotype frequencies were 100% C/G, 0% C/G, and 0% G/G, in perfect agreement with those predicted for Hardy-Weinberg equilibrium.

A frequency of 0.99 for the C allele (“q”) and 0.01 for the G allele (“p”) among African-American control individuals predicts genotype frequencies of 98.0% C/C, 2.0% C/G, and 0% G/G at Hardy-Weinberg equilibrium (p²+2pq+q²=1). The observed genotype frequencies were 97.7% C/G, 2.3% C/G, and 0% G/G, in excellent agreement with those predicted for Hardy-Weinberg equilibrium.

Using an allele-specific odds ratio of 1.5 or greater as a practical level of significance (Austin et al., discussed above), the following observations can be made.

For African-Americans with breast cancer the odds ratio for the G allele was 3.8 (95% CI, 0.2-62.8). Data were not sufficient to generate genotypic odds ratios of 1.5 or greater. These data suggest that the G allele acts in a co-dominant manner in this patient population. These data further suggest that the TGF-β1 gene is significantly associated with breast cancer in African-Americans, i.e. abnormal activity of the TGF-β1 gene predisposes African-Americans to breast cancer.

For African-American men with prostate cancer the odds ratio for the G allele was 2.0 (95% CI, 0.1-32.7). Data were not sufficient to generate genotypic odds ratios of 1.5 or greater. These data suggest that the G allele acts in a co-dominant manner in this patient population. These data further suggest that the TGF-β1 gene is significantly associated with prostate cancer in African-Americans, i.e. abnormal activity of the TGF-β1 gene predisposes African-American men to prostate cancer.

For African-Americans with atrial fibrillation but without valvular disease the odds ratio for the G allele was 5.8 (95% CI, 0.6-57.4). The odds ratio for the homozygote (G/G) was 0.5^{H} (95% CI, 0-8.4), while the odds ratio for the heterozygote (C/G) was 2.3^{H}(95% CI, 0-182.9). These data suggest that the G allele acts in a co-dominant manner in this patient population. These data further suggest that the TGF-β1 gene is significantly associated with Afib without valvular disease in African-Americans, i.e. abnormal activity of the TGF-β1 gene predisposes African-Americans to Afib without valvular disease.

For African-Americans with alcohol abuse the odds ratio for the G allele was 3.8 (95% CI, 0.3-42.8). The odds ratio for the homozygote (G/G) was 0.5^{H} (95% CI, 0-8.8), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with alcohol abuse in African-Americans, i.e. abnormal activity of the TGF-β1 gene predisposes African-Americans to alcohol abuse.

For African-Americans with anxiety the odds ratio for the G allele was 7.9 (95% CI, 0.9-72.9). The odds ratio for the homozygote (G/G) was 0.5^{H} (95% CI, 0-8), while the odds ratio for the heterozygote (C/G) was 3.0^{H} (95% CI, 0-228.7). These data suggest that the G allele acts in a co-dominant manner in this patient population. These data further suggest that the TGF-β1 gene is significantly associated with anxiety in African-Americans, i.e. abnormal activity of the TGF-β1 gene predisposes African-Americans to anxiety.

For Caucasians with anxiety the odds ratio for the G allele was 6.7^{H} (95% CI, 0.3-167.6). The odds ratio for the homozygote (G/G) was 0.4^{H} (95% CI, 0-7.5), while the odds ratio for the heterozygote (C/G) was 3.0^{H} (95% CI, 0-473.1). These data suggest that the G allele acts in a co-dominant manner in this patient population. These data further suggest that the TGF-β1 gene is significantly associated with anxiety in Caucasians, i.e. abnormal activity of the TGF-β1 gene predisposes Caucasians to anxiety.

For African-Americans with asthma the odds ratio for the G allele was 7.9 (95% CI, 0.9-72.9). The odds ratio for the homozygote (G/G) was 0.5^{H}(95% CI, 0-8), while the odds ratio for the heterozygote (C/G) was 3.0^{H} (95% CI, 0-228.7). These data suggest that the G allele acts in a co-dominant manner in this patient population. These data further suggest that the TGF-β1 gene is significantly associated with asthma in African-Americans, i.e. abnormal activity of the TGF-β1 gene predisposes African-Americans to asthma.

For African-Americans with cataracts due to HTN the odds ratio for the G allele was 7.9 (95% CI, 0.9-72.9). The odds ratio for the homozygote (G/G) was 0.5^{H} (95% CI, 0-8), while the odds ratio for the heterozygote (C/G) was 3.0^{H} (95% CI, 0-228.7). These data suggest that the G allele acts in a co-dominant manner in this patient population. These data further suggest that the TGF-β1 gene is significantly associated with cataracts due to HTN in African-Americans, i.e. abnormal activity of the TGF-β1 gene predisposes African-Americans to cataracts due to HTN.

For Caucasians with cataracts due to HTN the odds ratio for the G allele was 10.9 ^{H}(95% CI, 0.5-231.6). The odds ratio for the homozygote (G/G) was 0.4^{H} (95% CI, 0-7.5), while the odds ratio for the heterozygote (C/G) was 5.0^{H} (95% CI, 0-711.9). These data suggest that the G allele acts in a co-dominant manner in this patient population. These data further suggest that the TGF-β1 gene is significantly associated with cataracts due to HTN in Caucasians, i.e. abnormal activity of the TGF-β1 gene predisposes Caucasians to cataracts due to HTN.

For African-Americans with ESRD due to hypertension the odds ratio for the G allele was 2.0. (95% CI, 0.2-23.4), compared to African-Americans with hypertension only. The odds ratio for the homozygote (G/G) was _{1.0}^{H} (95% CI, 0.1-16.8), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with ESRD due to hypertension in African-Americans, i.e. abnormal activity of the TGF-β1 gene predisposes African-Americans to ESRD due to hypertension.

For African-Americans with cholecystectomy the odds ratio for the G allele was 1.9 (95% CI, 0.1-30.3). Data were not sufficient to generate genotypic odds ratios of 1.5 or greater. These data further suggest that the TGF-β1 gene is significantly associated with cholecystectomy in African-Americans, i.e. abnormal activity of the TGF-β1 gene predisposes African-Americans to cholecystectomy.

For African-Americans with colon cancer the odds ratio for the C allele was 1.6^{H} (95% CI, 0.1-39.9). Data were not sufficient to generate genotypic odds ratios of 1.5 or greater. These data further suggest that the TGF-β1 gene is significantly associated with colon cancer in African-Americans, i.e. abnormal activity of the TGF-β1 gene predisposes African-Americans to colon cancer.

For Caucasians with colon cancer the odds ratio for the G allele was 6.4^{H} (95% CI, 0.3-159.8). The odds ratio for the homozygote (G/G) was 0.5^{H} (95% CI, 0-7.9), while the odds ratio for the heterozygote (C/G) was 3.0^{H} (95% CI, 0-473.1). These data suggest that the G allele acts in a co-dominant manner in this patient population. These data further suggest that the TGF-β1 gene is significantly associated with colon cancer in Caucasians, i.e. abnormal activity of the TGF-β1 gene predisposes Caucasians to colon cancer.

For African-Americans with COPD the odds ratio for the G allele was 7.1 (95% CI, 0.7-70.1). The odds ratio for the homliozygote (G/G) was 0.4^{H}(95% CI, 0-6.9), while the odds ratio for the heterozygote (C/G) was 2.3^{H} (95% CL, 0-182.9). These data suggest that the G allele acts in a co-dominant manner in this patient population. These data further suggest that the TGF-β1 gene is significantly associated with COPD in African-Americans, i.e. abnormal activity of the TGF-β1 gene predisposes African-Americans to COPD.

For African-Americans with diabetic cardiomyopathy the odds ratio for the G allele was 2.1 (95% CI, 0.2-24.4), compared to African-Americans with MI due to NIDDM. The odds ratio for the homozygote (G/G) was 0.9^{H} (95% CI, 0.1-16), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with diabetic cardiomyopathy in African-Americans, i.e. abnormal activity of the TGF-β1 gene predisposes African-Americans to diabetic cardiomyopathy.

For African-Americans with DJD (osteoarthritis) the odds ratio for the G allele was 7.1(95% CI, 0.7-70.1). The odds ratio for the homozygote (G/G) was 0.4^{H} (95% CI, 0-6.9), while the odds ratio for the heterozygote (C/G) was 2.3^{H} (95% CI, 0-182.9). These data suggest that the G allele acts in a co-dominant manner in this patient population. These data further suggest that the TGF-β1 gene is significantly associated with DJD (osteoarthritis) in African-Americans, i.e. abnormal activity of the TGF-β1 gene predisposes African-Americans to DJD (osteoarthritis).

For African-Americans with ESRD and frequent de-clots the odds ratio for the G allele was 7.9 (95% CI, 0.9-72.9). Data were not sufficient to generate genotypic odds ratios of 1.5 or greater. These data further suggest that the TGF-β1 gene is significantly associated with ESRD and frequent de-clots in African-Americans, i.e. abnormal activity of the TGF-β1 gene predisposes African-Americans to ESRD and frequent de-clots.

For African-Americans with ESRD due to IDDM the odds ratio for the G allele was 3.8 (95% CI, 0.3-42.8). The odds ratio for the homozygote (G/G) was 0.5^{H} (95% CI, 0-8.8), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with ESRD due to IDDM in Africans-Americans, i.e. abnormal activity of the TGF-β1 gene predisposes African-Americans to ESRD due to IDDM.

For Caucasians with ESRD due to IDDM the odds ratio for the G allele was 5.8^{H} (95% CI, 0.2-146.2). The odds ratio for the honmozygote (G/G) was 0.5^{H} (95% CI, 0-8.6), while the odds ratio for the heterozygote (C/G) was 3.0^{H} (95% CI, 0-473.1). These data suggest that the G allele acts in a co-dominant manner in this patient population. These data furher suggest that the TGF-β1 gene is significantly associated with ESRD due to IDDM in Caucasians, i.e. abnormal activity of the TGF-β1 gene predisposes Caucasians to ESRD due to IDDM.

For African-Americans with ESRD due to FSGS the odds ratio for the G allele was 2.1 (95% CI, 0.1-34.8). Data were not sufficient to generate genotypic odds ratios of 1.5 or greater. These data further suggest that the TGF-β1 gene is significantly associated with ESRD due to FSGS in African-Americans, i.e. abnormal activity of the TGF-β1 gene predisposes African-Americans to ESRD due to FSGS.

For African-Americans with hypertensive cardiomyopathy the odds ratio for the G allele was 1.8 (95% CI, 0.2-20.4), compared to African-Americans with MI due to HTN. The odds ratio for the homozygote (G/G) was 1.1^{H} (95% CI, 0.1-19.3), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with hypertensive cardiomyopathy in African-Americans, i.e. abnormal activity of the TGF-β1 gene predisposes African-Americans to hypertensive cardiomyopathy.

For African-Americans with NIDDM the odds ratio for the G allele was 1.9 (95% CI, 0.1-30.3). Data were not sufficient to generate genotypic odds ratios of 1.5 or greater. These data further suggest that the TGF-β1 gene is significantly associated with NIDDM in African-Americans, i.e. abnormal activity of the TGF-β1 gene predisposes African-Americans to NIDDM.

For African-Americans with CVA due to NIDDM the odds ratio for the G allele was 2.0 (95% CI, 0.2-23.3), compared to African-Americans with NIDDM only. The odds ratio for the homozygote (G/G) was 1.0^{H} (95% CI, 0.1-16.7), while the odds ratio for the heterozygote (C/G) was 1.7^{H} (95% CI, 0-137.4). These data suggest that the G allele acts in a co-dominant manner in this patient population. These data further suggest that the TGF-β1 gene is significantly associated with CVA due to NIDDM in African-Americans, i.e. abnormal activity of the TGF-β1 gene predisposes African-Americans to CVA due to NIDDM.

For African-Americans with seizure disorder the odds ratio for the G allele was 6.1 (95% CI, 0.6-60.1). The odds ratio for the homozygote (G/G) was 0.5^{H} (95% CI, 0-8), while the odds ratio for the heterozygote (C/G) was 2.3^{H} (95% CI, 0-182.9). These data suggest that the G allele acts in a co-dominant manner in this patient population. These data further suggest that the TGF-β1 gene is significantly associated with seizure disorder in African-Americans, i.e. abnormal activity of the TGF-β1 gene predisposes African-Americans to seizure disorder.

According to Matlnspector (GENOMATIX; see above for URL and reference), the C2164→G transversion is predicted to have the following effects on transcription of the TGF-β1 gene:

a. Disruption of a putative FSE2 site (nucleotides #216 to #224) in the TGF-β1 promoter, approximately 2kb upstream (5′) of the transcription initiation site. The TGF-β1 promoter has two FSE2 sites; the second one is located approximately 600 bases downstream from the first site (at nucleotides #807-816). FSE2 sites are potent negative transcriptional regulatory sites; disruption of a site is thus expected to result in increased transcription of the TGF-β1 gene. Assuming that mRNA stability, translational efficiency, etc. are unchanged, this SNP is expected to result in increased cellular production and secretion of TGF-β1.

b. Disruption of a potential GKLF (gut-enriched Krueppel-like factor) site beginning at nucleotide #211 according to numbering on the (+) strand. The binding site is actually located on the (−) strand, and consists of the complement to the sequence 5′-CCYYTYYYTYNTTY-3′ (SEQ ID NO: 6). The SNP replaces the underlined Y (C or T) with a G. GKLF sites occur relatively frequently, 4.76 matches per 1000 base pairs of random genomic sequence in vertebrates.

GKLF is a transcriptional activator, so disruption of its binding site in the TGF-β1 promoter should result in a lower rate of TGF-β1 transcription, and ultimately a lower level of TGF-β1 produced in tissues.

### Example 3

### G to A Transition at Position 563 of the Human TGF-β1 Promoter

TABLE 7
ALLELE FREQUENCY

| Disease | Race | CHROMOSOMES | N | G | N | A |
|---|---|---|---|---|---|---|
| Controls | African-American | 90 | 87 | 96.7% | 3 | 3.3% |
| | Caucasian | 86 | 78 | 90.7% | 8 | 9.3% |
| Colon cancer | African-American | 48 | 47 | 97.9% | 1 | 2.1% |
| | Caucasian | 48 | 43 | 89.6% | 5 | 10.4% |
| Lung cancer | African-American | 40 | 39 | 97.5% | 1 | 2.5% |
| | Caucasian | 44 | 40 | 90.9% | 4 | 9.1% |
| Hypertension | African-American | 48 | 46 | 95.8% | 2 | 4.2% |
| | Caucasian | 44 | 35 | 79.5% | 9 | 20.5% |
| ASPVD due to HTN | African-American | 50 | 50 | 100.0% | 0 | 0.0% |
| | Caucasian | 50 | 47 | 94.0% | 3 | 6.0% |
| CVA due to HTN | African-American | 48 | 39 | 81.3% | 9 | 18.8% |
| | Caucasian | 46 | 41 | 89.1% | 5 | 10.9% |
| Cataracts due to HTN | African-American | 48 | 47 | 97.9% | 1 | 2.1% |
| | Caucasian | 44 | 44 | 100.0% | 0 | 0.0% |
| HTN CM | African-American | 48 | 36 | 75.0% | 12 | 25.0% |
| | Caucasian | 46 | 37 | 80.4% | 9 | 19.6% |
| MI due to HTN | African-American | 42 | 41 | 97.6% | 1 | 2.4% |
| | Caucasian | 42 | 37 | 88.1% | 5 | 11.9% |
| NIDDM | African-American | 40 | 40 | 100.0% | 0 | 0.0% |
| ASPVD due to NIDDM | African-American | 42 | 41 | 97.6% | 1 | 2.4% |
| | Caucasian | 44 | 38 | 86.4% | 6 | 13.6% |
| CVA due to NIDDM | African-American | 48 | 48 | 100.0% | 0 | 0.0% |
| | Caucasian | 46 | 40 | 87.0% | 6 | 13.0% |
| ESRD due to NIDDM | African-American | 42 | 39 | 92.9% | 3 | 7.1% |
| | Caucasian | 46 | 42 | 93.1% | 4 | 8.7% |
| Ischemic CM | African-American | 48 | 48 | 100.0% | 0 | 0.0% |
| | Caucasian | 42 | 37 | 88.1% | 5 | 11.9% |
| Ischemic CM with NIDDM | African-American | 48 | 48 | 100.0% | 0 | 0.0% |
| | Caucasian | 46 | 41 | 89.1% | 5 | 10.9% |
| MI due to NIDDM | African-American | 48 | 45 | 93.8% | 3 | 6.3% |
| | Caucasian | 48 | 45 | 93.8% | 3 | 6.3% |
| Afib without valvular disease | African-American | 48 | 48 | 100.0% | 0 | 0.0% |
| | Caucasian | 48 | 47 | 97.9% | 1 | 2.1% |
| Alcohol abuse | African-American | 48 | 48 | 100.0% | 0 | 0.0% |
| | Caucasian | 48 | 44 | 91.7% | 4 | 8.3% |
| Anxiety | African-American | 48 | 47 | 97.9% | 1 | 2.1% |
| | Caucasian | 40 | 36 | 90.0% | 4 | 10.0% |
| Asthma | African-American | 48 | 48 | 100.0% | 0 | 0.0% |
| | Caucasian | 48 | 42 | 87.5% | 6 | 12.5% |
| COPD | African-American | 40 | 38 | 95.0% | 2 | 5.0% |
| | Caucasian | 46 | 40 | 87.0% | 6 | 13.0% |
| Cholecystectomy | African-American | 46 | 43 | 93.5% | 3 | 6.5% |
| | Caucasian | 48 | 43 | 89.6% | 5 | 10.4% |
| DJD | African-American | 40 | 39 | 97.5% | 1 | 2.5% |
| | Caucasian | 40 | 36 | 90.0% | 4 | 10.0% |
| ESRD and frequent de-clots | African-American | 48 | 48 | 100.0% | 0 | 0.0% |
| | Caucasian | 44 | 42 | 95.5% | 2 | 4.5% |
| ESRD due to FSGS | African-American | 42 | 40 | 95.2% | 2 | 4.8% |
| | Caucasian | 44 | 39 | 88.6% | 5 | 11.4% |
| ESRD due to IDDM | African-American | 48 | 47 | 97.9% | 1 | 2.1% |
| | Caucasian | 48 | 43 | 89.6% | 5 | 10.4% |
| Seizure disorder | African-American | 48 | 46 | 95.8% | 2 | 4.2% |
| | Caucasian | 46 | 43 | 93.5% | 3 | 6.5% |

TABLE 8
GENOTYPE FREQUENCY

| Disease | Race | People | N | G/G | N | G/A | N | A/A |
|---|---|---|---|---|---|---|---|---|
| Controls | African-American | 45 | 43 | 95.6% | 1 | 2.2% | 1 | 2.2% |
| | Caucasian | 43 | 35 | 81.4% | 8 | 18.6% | 0 | 0.0% |
| Colon cancer | African-American | 24 | 23 | 95.8% | 1 | 4.2% | 0 | 0.0% |
| | Caucasian | 24 | 20 | 83.3% | 3 | 12.5% | 1 | 4.2% |
| Lung Cancer | African-American | 20 | 19 | 95.0% | 1 | 5.0% | 0 | 0.0% |
| | Caucasian | 22 | 18 | 81.8% | 4 | 18.2% | 0 | 0.0% |
| ASPVD due to HTN | African-American | 25 | 25 | 100.0% | 0 | 0.0% | 0 | 0.0% |
| | Caucasian | 25 | 22 | 88.0% | 3 | 12.0% | 0 | 0.0% |
| CVA due to HTN | African-American | 24 | 15 | 62.5% | 9 | 37.5% | 0 | 0.0% |
| | Caucasian | 23 | 18 | 78.3% | 5 | 21.7% | 0 | 0.0% |
| Cataracts due to HTN | African-American | 24 | 23 | 95.8% | 1 | 4.2% | 0 | 0.0% |
| | Caucasian | 22 | 22 | 100.0% | 0 | 0.0% | 0 | 0.0% |
| HTN CM | African-American | 24 | 12 | 50.0% | 12 | 50.0% | 0 | 0.0% |
| | Caucasian | 23 | 14 | 60.9% | 9 | 39.1% | 0 | 0.0% |
| MI due to HTN | African-American | 21 | 20 | 95.2% | 1 | 4.8% | 0 | 0.0% |
| | Caucasian | 21 | 16 | 76.2% | 5 | 23.8% | 0 | 0.0% |
| NIDDM | African-American | 20 | 20 | 100.0% | 0 | 0.0% | 0 | 0.0% |
| ASPVD due to NIDDM | African-American | 21 | 20 | 95.2% | 1 | 4.8% | 0 | 0.0% |
| | Caucasian | 22 | 16 | 72.7% | 6 | 27.3% | 0 | 0.0% |
| CVA due to NIDDM | African-American | 24 | 24 | 100.0% | 0 | 0.0% | 0 | 0.0% |
| | Caucasian | 23 | 17 | 73.9% | 6 | 26.1% | 0 | 0.0% |
| ESRD due to NIDDM | African-American | 21 | 19 | 90.5% | 1 | 4.8% | 1 | 4.8% |
| | Caucasian | 23 | 19 | 82.6% | 4 | 17.4% | 0 | 0.0% |
| Ischemic CM | African-American | 24 | 24 | 100.0% | 0 | 0.0% | 0 | 0.0% |
| | Caucasian | 21 | 16 | 76.2% | 5 | 23.8% | 0 | 0.0% |
| Ischemic CM with NIDDM | African-American | 24 | 24 | 100.0% | 0 | 0.0% | 0 | 0.0% |
| | Caucasian | 23 | 18 | 78.3% | 5 | 21.7% | 0 | 0.0% |
| MI due to NIDDM | African-American | 24 | 21 | 87.5% | 3 | 12.5% | 0 | 0.0% |
| | Caucasian | 24 | 21 | 87.5% | 3 | 12.5% | 0 | 0.0% |
| Afib without valvular disease | African-American | 24 | 24 | 100.0% | 0 | 0.0% | 0 | 0.0% |
| | Caucasian | 24 | 23 | 95.8% | 1 | 4.2% | 0 | 0.0% |
| Alcohol abuse | African-American | 24 | 24 | 100.0% | 0 | 0.0% | 0 | 0.0% |
| | Caucasian | 24 | 20 | 83.3% | 4 | 16.7% | 0 | 0.0% |
| Anxiety | African-American | 24 | 23 | 95.8% | 1 | 4.2% | 0 | 0.0% |
| | Caucasian | 20 | 16 | 80.0% | 4 | 20.0% | 0 | 0.0% |
| Asthma | African-American | 24 | 24 | 100.0% | 0 | 0.0% | 0 | 0.0% |
| | Caucasian | 24 | 19 | 79.2% | 4 | 16.7% | 1 | 4.2% |
| COPD | African-American | 20 | 18 | 90.0% | 2 | 10.0% | 0 | 0.0% |
| | Caucasian | 23 | 18 | 78.3% | 4 | 17.4% | 1 | 4.3% |
| Cholecystectomy | African-American | 23 | 20 | 87.0% | 3 | 13.0% | 0 | 0.0% |
| | Caucasian | 24 | 19 | 79.2% | 5 | 20.8% | 0 | 0.0% |
| DJD | African-American | 20 | 19 | 95.0% | 1 | 5.0% | 0 | 0.0% |
| | Caucasian | 20 | 16 | 80.0% | 4 | 20.0% | 0 | 0.0% |
| ESRD and frequent de-clots | African-American | 24 | 24 | 100.0% | 0 | 0.0% | 0 | 0.0% |
| | Caucasian | 22 | 20 | 90.9% | 2 | 9.1% | 0 | 0.0% |
| ESRD due to FSGS | African-American | 21 | 19 | 90.5% | 2 | 9.5% | 0 | 0.0% |
| | Caucasian | 22 | 17 | 77.3% | 5 | 22.7% | 0 | 0.0% |
| ESRD due to IDDM | African-American | 24 | 23 | 95.8% | 1 | 4.2% | 0 | 0.0% |
| | Caucasian | 24 | 19 | 79.2% | 5 | 20.8% | 0 | 0.0% |
| Seizure disorder | African-American | 24 | 22 | 91.7% | 2 | 8.3% | 0 | 0.0% |
| | Caucasian | 23 | 21 | 91.3% | 1 | 4.3% | 1 | 4.3% |

Allele-Specific Odds Ratios

The susceptibility allele is indicated below, as well as the odds ratio (OR). Haldane's correction was used if the denominator is zero, and so indicated (“H”). If the odds ratio (OR) is ≧1.5, the 95% confidence interval (C.I.) is also given. An odds ratio of 1.5 is chosen as the threshold of significance based on the recommendation of Austin et al. in Epidemiol. Rev. 16:65-76, 1994.

“. . . [E]pidemiology in general and case-control studies in particular are not well suited for detecting weak associations (odds ratios <1.5).” Id. at 66. Odds ratios of 1.5 or higher are high-lighted below.

TABLE 9
ALLELE-SPECIFIC ODDS RATIOS

| | | | | Lower | Upper | |
|---|---|---|---|---|---|---|
| | | | | Limit | Limit | |
| | | Risk | Odds | 95% | 95% | |
| Disease | Race | Allele | Ratio | CI | CI | Haldane |
| Colon cancer | African-American | C | 1.6 | 0.2 | 16.0 | |
| | Caucasian | A | 1.1 | 0.3 | 3.7 | |
| Lung cancer | African-American | G | 1.3 | 0.1 | 13.3 | |
| | Caucasian | G | 1.0 | 0.3 | 3.6 | |
| Hypertension | African-American | A | 1.3 | 0.2 | 7.8 | |
| | Caucasian | A | 2.5 | 0.9 | 7.0 | |
| ASPVD due to HTN* | African-American | G | 5.4 | 0.3 | 116.1 | H |
| | Caucasian | G | 4.0 | 1.0 | 16.0 | |
| CVA due to HTN* | African-American | A | 5.3 | 1.1 | 26.0 | |
| | Caucasian | G | 2.1 | 0.6 | 6.9 | |
| Cataracts due to HTN* | African-American | G | 1.6 | 0.2 | 16.0 | |
| | Caucasian | G | 9.6 | 0.5 | 171.0 | H |
| HTN CM*¹ | African-American | A | 13.7 | 1.7 | 110.3 | |
| | Caucasian | A | 1.8 | 0.6 | 5.9 | |
| MI due to HTN* | African-American | G | 1.8 | 0.2 | 20.4 | |
| | Caucasian | G | 1.9 | 0.6 | 6.2 | |
| NIDDM | African-American | G | 3.2 | 0.2 | 64.2 | H |
| ASPVD due to NIDDM*² | African-American | A | 2.9 | 0.1 | 74.0 | H |
| CVA due to NIDDM*² | African-American | G | 1.0 | — | — | |
| ESRD due to NIDDM*² | African-American | A | 7.2 | 0.4 | 143.5 | H |
| Ischemic CM with NIDDM*³ | African-American | G | 7.5 | 0.4 | 148.5 | H |
| | Caucasian | A | 1.8 | 0.4 | 8.1 | |
| MI due to NIDDM*² | African-American | A | 6.2 | 0.3 | 124.3 | H |
| Afib without valvular disease | African-American | G | 3.9 | 0.2 | 76.7 | H |
| | Caucasian | G | 4.8 | 0.6 | 39.8 | |
| Alcohol abuse | African-American | G | 3.9 | 0.2 | 76.7 | H |
| | Caucasian | G | 1.1 | 0.3 | 4.0 | |
| Anxiety | African-American | G | 1.6 | 0.2 | 16.0 | |
| | Caucasian | A | 1.1 | 0.3 | 3.8 | |
| Asthma | African-American | G | 3.9 | 0.2 | 76.7 | H |
| | Caucasian | A | 1.4 | 0.5 | 4.3 | |
| COPD | African-American | A | 1.5 | 0.2 | 9.5 | |
| | Caucasian | A | 1.5 | 0.5 | 4.5 | |
| Cholecystectomy | African-American | A | 2.0 | 0.4 | 10.4 | |
| | Caucasian | A | 1.1 | 0.3 | 3.7 | |
| DJD | African-American | G | 1.3 | 0.1 | 13.3 | |
| | Caucasian | A | 1.1 | 0.3 | 3.8 | |
| ESRD and frequent de-clots | African-American | G | 3.9 | 0.2 | 76.7 | H |
| | Caucasian | G | 2.2 | 0.4 | 10.6 | |
| ESRD due to FSGS | African-American | A | 1.5 | 0.2 | 9.0 | |
| | Caucasian | A | 1.3 | 0.4 | 4.1 | |
| ESRD due to IDDM | African-American | G | 1.6 | 0.2 | 16.0 | |
| | Caucasian | A | 1.1 | 0.3 | 3.7 | |
| Seizure disorder | African-American | A | 1.3 | 0.2 | 7.8 | |
| | Caucasian | G | 1.5 | 0.4 | 5.8 | |

*Compared to HTN alone.
*¹Compared to MI with HTN.
*²Compared to NIDDM alone.
*³Compared to MI with NIDDM.

Genotype-Specific Odds Ratios

The susceptibility allele (S) is indicated; the alternative allele at this locus is The susceptibility allele (S) is indicated; the alternative allele at this locus is defined as the protective allele (P). Also presented is the odds ratio (OR) for the SS and SP genotypes; the odds ratio for the PP genotype is defined as 1, since it serves as the reference group, and is not presented separately. For odds ratios ≧1.5, the 95% confidence interval (C.I.) is also given in parentheses. An odds ratio of 1.5 was chosen as the threshold of significance based on the recommendation of Austin et al. in Epidemiol. Rev. 16:65-76, 1994. “[E]pidemiology in general and case-control studies in particular are not well suited for detecting weak associations (odds ratios <1.5).” Id. at 66.

Odds ratios of 1.5 or higher are high-lighted below. Where Haldane's zero cell correction was used, the odds ratio is so indicated with a superscript “H”.

TABLE 10
GENOTYPE-SPECIFIC ODDS RATIOS

| | | RISK | SS | | SP | |
|---|---|---|---|---|---|---|
| Disease | Race | ALLELE | O.R. | HALDANE | O.R. | HALDANE |
| Colon cancer | African-American | G | 1.6 | H | 3.0 | H |
| | Caucasian | A | 0.0 | | 0.0 | |
| Lung cancer | African-American | G | 1.3 | H | 3.0 | H |
| | Caucasian | G | 0.5 | H | 0.5 | H |
| Hypertension | African-American | A | 1.6 | H | 5.0 | H |
| | Caucasian | A | 0.0 | | 0.0 | |
| ASPVD due to HTN* | African-American | G | 1.1 | H | 0.2 | H |
| | Caucasian | G | 4.7 | H | 1.4 | H |
| CVA due to HTN* | African-American | A | 0.7 | H | 3.8 | H |
| | Caucasian | G | 3.8 | H | 2.2 | H |
| Cataracts due to HTN* | African-American | G | 1.6 | H | 3.0 | H |
| | Caucasian | G | 0.6 | H | 0.1 | H |
| HTN CM*¹ | African-American | A | 0.6 | H | 8.3 | H |
| | Caucasian | A | 0.9 | H | 1.7 | H |
| MI due to HTN* | African-American | G | 0.9 | H | 0.6 | H |
| | Caucasian | G | 3.4 | H | 2.2 | H |
| NIDDM | African-American | G | 1.4 | H | 1.0 | H |
| ASPVD due to NIDDM*² | African-American | A | 1.0 | H | 3.0 | H |
| CVA due to NIDDM*² | African-American | G | 1.2 | H | 1.0 | H |
| ESRD due to NIDDM*² | African-American | A | 0.0 | | 1.0 | H |
| Ischemic CM with NIDDM*³ | African-American | G | 1.1 | H | 0.1 | H |
| | Caucasian | A | 0.9 | H | 1.6 | H |
| MI due to NIDDM*² | African-American | A | 1.0 | H | 7.0 | H |
| Afib without valvular disease | African-American | G | 1.7 | H | 1.0 | H |
| | Caucasian | G | 0.7 | H | 0.2 | H |
| Alcohol abuse | African-American | G | 1.7 | H | 1.0 | H |
| | Caucasian | G | 0.6 | H | 0.5 | H |
| Anxiety | African-American | G | 1.6 | H | 3.0 | H |
| | Caucasian | A | 0.5 | H | 0.5 | H |
| Asthma | African-American | G | 1.7 | H | 1.0 | H |
| | Caucasian | A | 0.0 | | 0.0 | |
| COPD | African-American | A | 1.3 | H | 5.0 | H |
| | Caucasian | A | 0.0 | | 0.0 | |
| Cholecystectomy | African-American | A | 1.4 | H | 7.0 | H |
| | Caucasian | A | 0.5 | H | 0.6 | H |
| DJD | African-American | G | 1.3 | H | 3.0 | H |
| | Caucasian | A | 0.5 | H | 0.5 | H |
| ESRD and frequent de-clots | African-American | G | 1.7 | H | 1.0 | H |
| | Caucasian | G | 0.6 | H | 0.3 | H |
| ESRD due to FSGS | African-American | A | 1.3 | H | 5.0 | H |
| | Caucasian | A | 0.5 | H | 0.6 | H |
| ESRD due to IDDM | African-American | G | 1.6 | H | 3.0 | H |
| | Caucasian | A | 0.5 | H | 0.6 | H |
| Seizure disorder | African-American | A | 1.6 | H | 5.0 | H |
| | Caucasian | G | 0.0 | | 0.0 | |

*Compared to HTN alone.
*¹Compared to MI with HTN.
*²Compared to NIDDM alone.
*³Compared to MI with NIDDM.

PCR and sequencing were conducted as described in Example 1. The primers used were those in Example 1. The control samples were in good agreement with Hardy-Weinberg equilibrium, as follows:

A frequency of 0.967 for the G allele (“q”) and 0.033 for the A allele (“p”) among African-American control individuals predicts genotype frequencies of 93.5% G/G, 6.4% G/A, and 0.1% A/A at Hardy-Weinberg equilibrium (p²+2pq+q²=1). The observed genotype frequencies were 95.6% G/G, 2.2% G/A, and 2.2% A/A, in good agreement with those predicted for Hardy-Weinberg equilibrium.

A frequency of 0.91 for the G allele (“q”) and 0.09 for the A allele (“p”) among Caucasian control individuals predicts genotype frequencies of 82.8% G/G, 16.4% G/A, and 0.8% A/A at Hardy-Weinberg equilibrium (p²+2pq+q²=1). The observed genotype frequencies were 81.4% G/G, 18.6% G/A, and 0% A/A, in excellent agreement with those predicted for Hardy-Weinberg equilibrium.

Using an allele-specific odds ratio of 1.5 or greater as a practical level of significance (see Austin et al., discussed above), the following observations can be made.

For African-Americans with atrial fibrillation but without valvular disease the odds ratio for the G allele was 3.9^{H} (95% CI, 0.2-76.7). The odds ratio for the homozygote (G/G) was 1.7^{H} (95% CI, 0.1-28.7), while the odds ratio for the heterozygote (G/A) was 1.0^{H} (95% CI, 0-92.4). These data suggest that the G allele acts in a recessive manner in this patient population. These data further suggest that the TGF-β1 gene is significantly associated with Afib without valvular disease in African-Americans, i.e. abnormal activity of the TGF-β1 gene predisposes African-Americans to Afib without valvular disease.

For Caucasians with atrial fibrillation but without valvular disease the odds ratio for the G allele was 4.8 (95% CI, 0.6-39.8). Data were not sufficient to generate genotypic odds ratios of 1.5 or greater. These data further suggest that the TGF-β1 gene is significantly associated with Afib without valvular disease in Caucasians, i.e. abnormal activity of the TGF-β1 gene predisposes Caucasians to Afib without valvular disease.

For African-Americans with a history of alcohol abuse the odds ratio for the G allele was 3.9^{H} (95% CI, 0.2-76.7). The odds ratio for the homozygote (G/G) was 1.7^{H} (95% CI, 0.1-28.7), while the odds ratio for the heterozygote (G/A) was 1.0^{H} (95% CI, 0-92.4). These data suggest that the G allele acts in a recessive manner in this patient population. These data further suggest that the TGF-β1 gene is significantly associated with alcohol abuse in African-Americans, i.e. abnormal activity of the TGF-β1 gene predisposes African-Americans toalcohol abuse.

For African-Americans with anxiety the odds ratio for the G allele was 1.6 (95% CI, 0.2-16). The odds ratio for the homozygote (G/G) was 1.6^{H} (95% CI, 0.1-27.5), while the odds ratio for the heterozygote (G/A) was 3.0^{H} (95% CI, 0.1-151.2). These data suggest that the G allele acts in a co-dominant manner in this patient population. These data further suggest that the TGF-β1 gene is significantly associated with anxiety in African-Americans, i.e. abnormal activity of the TGF-β1 gene predisposes African-Americans to anxiety.

For African-Americans with ASPVD due to NIDDM the odds ratio for the A allele was 2.9^{H} (95% CI, 0.1-74), compared to African-Americans with NIDDM alone. The odds ratio for the homozygote (A/A) was 1.0^{H} (95% CI, 0.1-17.7), while the odds ratio for the heterozygote (G/A) was 3.0^{H} (95% CI, 0-473.1). These data suggest that the A allele acts in a co-dominant manner in this patient population. These data further suggest that the TGF-β1 genie is significantly associated with ASPVD due to NIDDM in African-Americans, i.e. abnormal activity of the TGF-β1 gene predisposes African-Americans to ASPVD due to NIDDM.

For African-Americans with asthma the odds ratio for the G allele was 3.9^{H} (95% CI, 0.2-76.7). The odds ratio for the homozygote (G/G) was 1.7^{H} (95% CI, 0.1-28.7), while the odds ratio for the heterozygote (G/A) was 1.0^{H} (95% CI, 0-92.4). These data suggest that the G allele acts in a recessive manner in this patient population. These data further suggest that the TGF-β1 gene is significantly associated with asthma in African-Americans, i.e. abnormal activity of the TGF-β1 gene predisposes African-Americans to asthma.

For African-Americans with cataracts due to HTN the odds ratio for the G allele was 1.6 (95% CI, 0.2-16). The odds ratio for the homozygote (G/G) was 1.6^{H} (95% CI, 0.1-27.5), while the odds ratio for the heterozygote (G/A) was 3.0^{H} (95% CI, 0.1-151.2). These data suggest that the G allele acts in a co-dominant manner in this patient population. These data further suggest that the TGF-β1 gene is significantly associated with cataracts due to HTN in African-Americans, i.e. abnormal activity of the TGF-β1 gene predisposes African-Americans to cataracts due to HTN.

For Caucasians with cataracts due to HTN the odds ratio for the G allele was 9.6^{H} (95% CI, 0.5-171). Data were not sufficient to generate genotypic odds ratios of 1.5 or greater. These data further suggest that the TGF-β1 gene is significantly associated with cataracts due to HTN in Caucasians, i.e. abnormal activity of the TGF-β1 gene predisposes Caucasians to cataracts due to HTN.

For African-Americans who had undergone a cholecystectomy the odds ratio for the A allele was 2.0 (95% CI, 0.4-10.4). The odds ratio for the homozygote (A/A) was 1.4^{H} (95% CI, 0.1-24.1), while the odds ratio for the heterozygote (G/A) was 7.0^{H} (95% CI, 0.2-291.4). These data suggest that the A allele acts in a co-dominant manner in this patient population. These data further suggest that the TGF-β1 gene is significantly associated with cholecystectomy in African-Americans, i.e. abnormal activity of the TGF-β1 gene predisposes African-Americans to cholecystectomy.

For African-Americans with colon cancer the odds ratio for the G allele was 1.6 (95% CI, 0.2-16). The odds ratio for the homozygote (G/G) was 1.6^{H} (95% CI, 0.1-27.5), while the odds ratio for the heterozygote (G/A) was 3.0^{H} (95% CI, 0.1-151.2). These data suggest that the G allele acts in a co-dominant manner in this patient population. These data further suggest that the TGF-β1 gene is significantly associated with colon cancer in African-Americans, i.e. abnormal activity of the TGF-β1 gene predisposes African-Americans to colon cancer.

For African-Americans with diabetic cardiomyopathy the odds ratio for the G allele was 7.5^{H} (95% CI, 0.4-148.5), compared to African-Americans with MI due to NIDDM. Data were not sufficient to generate genotypic odds ratios of 1.5 or greater. These data further suggest that the TGF-β1 gene is significantly associated with diabetic cardiomyopathy in African-Americans, i.e. abnormal activity of the TGF-β1 gene predisposes African-Americans to diabetic cardiomyopathy.

For Caucasians with diabetic cardiomyopathy the odds ratio for the A allele was 1.8 (95% CI, 0.4-8.1), compared to Caucasians with MI due to NIDDM. The odds ratio for the homozygote (T/T) was 0.9^{H} (95% CI, 0-15.2), while the odds ratio for the heterozygote (G/A) was 1.6^{H} (95% CI, 0-99). These data suggest that the A allele acts in a co-dominant manner in this patient population. These data further suggest that the TGF-β1 gene is significantly associated with diabetic cardiomyopathy in Caucasians, i.e. abnormal activity of the TGF-β1 gene predisposes Caucasians to diabetic cardiomyopathy.

For African-Americans with ESRD and frequent de-clots the odds ratio for the G allele was 3.9^{H} (95% CI, 0.2-76.7). The odds ratio for the homozygote (G/G) was 1.7^{H} (95% CI, 0.1-28.7), while the odds ratio for the heterozygote (G/A) was 1.0^{H} (95% CI, 0-92.4) These data suggest that the G allele acts in a recessive manner in this patient population. These data further suggest that the TGF-β1 gene is significantly associated with ESRD and frequent de-clots in African-Americans, i.e. abnormal activity of the TGF-β1 gene predisposes African-Americans to ESRD and frequent de-clots.

For Caucasians with ESRD and frequent de-clots the odds ratio for the G allele was 2.2 (95% CI, 0.4-10.6). Data were not sufficient to generate genotypic odds ratios of 1.5 or greater. These data further suggest that the TGF-β1 gene is significantly associated with ESRD and frequent de-clots in Caucasians, i.e. abnormal activity of the TGF-β1 gene predisposes Caucasians to ESRD and frequent de-clots.

For African-Americans with ESRD due to IDDM the odds ratio for the G allele was 1.6 (95% CI, 0.2-16). The odds ratio for the homozygote (G/G)was 1.6^{H} (95% CI, 0.1-27.5), while the odds ratio for the heterozygote (G/A) was 3.0^{H} (95% CI, 0.1-151.2). These data suggest that the G allele acts in a co-dominant manner in this patient population. These data further suggest that the TGF-β1 gene is significantly associated with ESRD due to IDDM in African-Americans, i.e. abnormal activity of the TGF-β1gene predisposes African-Americans to ESRD due to IDDM.

For African-Americans with ESRD due to NIDDM the odds ratio for the A allele was 7.2^{H} (95% CI, 0.4-143.5), compared to African-Americans with NIDDM only. Data were not sufficient to generate genotypic odds ratios of 1.5 or greater. These data further suggest that the TGF-β1 gene is significantly associated with ESRD due to NIDDM in African-Americans, i.e. abnormal activity of the TGF-β1 gene predisposes African-Americans to ESRD due to NIDDM.

For African-Americans with hypertensive cardiomyopathy the odds ratio for the A allele was 13.7 (95% CI, 1.7-110.3), compared to African-Americans with MI due to HTN. The odds ratio for the homozygote (A/A) was 0.6^{H} (95% CI, 0-11), while the odds ratio for the heterozygote (G/A) was 8.3^{H} (95% CI, 0.1-596.1). These data suggest that the A allele acts in a co-dominant manner in this patient population. These data further suggest that the TGF-β1 gene is significantly associated with hypertensive cardiomyopathy in African-Americans, i.e. abnormal activity of the TGF-β1 gene predisposes African-Americans to hypertensive cardiomyopathy.

For Caucasians with hypertensive cardiomyopathy the odds ratio for the A allele was 1.8 (95% CI, 0.6-5.9), compared to Caucasians with MI due to HTN. The odds ratio for the homozygote (A/A) was 0.9 H(95% CI, 0-16), while the odds ratio for the heterozygote (G/A) was 1.7^{H} (95% CI, 0-100). These data suggest that the A allele acts in a co-dominant manner in this patient population. These data further suggest that the TGF-β1 gene is significantly associated with hypertensive cardiomyopathy in Caucasians,

i.e. abnormal activity of the TGF-β1 gene predisposes Caucasians to hypertensive cardiomyopathy.

For African-Americans with NIDDM the odds ratio for the G allele was 3.2^{H} (95% CI, 0.2-64.2). Data were not sufficient to generate genotypic odds ratios of 1.5 or greater. These data further suggest that the TGF-β1 gene is significantly associated with NIDDM in African-Americans, i.e. abnormal activity of the TGF-β1 gene predisposes African-Americans to NIDDM.

For African-Americans with MI due to NIDDM the odds ratio for the A allele was 6.2^{H} (95% CI, 0.3-124.3), compared to African-Americans with NIDDM only. The odds ratio for the homozygote (A/A) was 1.0^{H} (95% CI, 0.1-18.5), while the odds ratio for the heterozygote (G/A) was 7.0^{H} (95% CI, 0.1-953.3). These data suggest that the A allele acts in a co-dominant manner in this patient population. These data further suggest that the TGF-β1 gene is significantly associated with MI due to NIDDM in African-Americans, i.e. abnormal activity of the TGF-β1 gene predisposes African-Americans to MI due to NIDDM. According to Mathispector (GENOMATIX; see above for URL and reference), the G563→A transition disrupts a binding sequence for the ubiquitous transcriptional activator cAMP Responsive-Element Binding protein (CREB; Paca-Uccaralerthun S., et al., Mol Cell Biol 14:456-462; 1994). The sequence, which is located on the antisense strand, corresponds to bases 559-570 on the (+) strand; its consensus sequence is 5′-NNRCGTCANCNN-3′. The wildtype sequence contained in bases 559-570 is 98% similar to the CREB site consensus (a weighted matrix of known vertebrate CREB binding sites; abbreviated as CREB_{—}02 in GENOMATIX), but this similarity is decreased by the G563→A SNP.

TGFβ is a powerful extracellular signaling polypeptide that is involved in embryonic development, and then later in life as a growth inhibitor. The TGFβ signal is propagated when it binds to a cell-surface receptor; this receptor facilitates phosphorylation of an intracellular molecule/complex (known as a second messenger) that then directs the signal to specific compartments of the cell. The most relevant effects of the signalling cascade are seen within the nucleus, where the second messenger, or some molecule downstream in its pathway, activates transcriptional factors. CREB is one such transcriptional factor, whose corresponding second messenger is cAMP. The presence of such a binding site within the TFGβ promoter region would imply that a cAMP-dependent signalling process is involved in the control of TGFβ expression. Although a small adjustment in the expression of TGFβ may be expected from the G563→A SNP, this would be consistent with the late, prolapsed (i.e.- not acute) onset of many of the diseases discussed in this application. Disease processes linked to this SNP may be linked to long-term depression of cell growth inhibition.

TABLE 11

| | | | Reference | | |
|---|---|---|---|---|---|
| Gene | Region | Location | Type | Variant | SEQ ID |
| TGF-β1 | Promoter | 216 | C | G | 1 |
| | | 563 | G | A | 1 |

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for diagnosing a genetic susceptibility for a disease, condition, or disorder in a subject comprising:
obtaining a biological sample containing nucleic acid from said subject; and analyzing said nucleic acid to detect the presence or absence of a single nucleotide polymorphism in the TGF-β1 gene, wherein said single nucleotide polymorphism is associated with a genetic predisposition for a disease, condition or disorder selected from the group consisting of breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder.

2. The method of claim 1, wherein the gene TGF-β1 comprises SEQ ID NO: 1.

3. The method of claim 1, wherein said nucleic acid is DNA, RNA, cDNA or mRNA.

4. The method of claim 2, wherein said single nucleotide polymorphism is located at position 216 or 563 of SEQ ID NO: 1.

5. The method of claim 4, wherein said single nucleotide polymorphism is selected from the group consisting of C216->G and G563->A and the complements thereof namely G216->C and C563->T.

6. The method of claim 1, wherein said analysis is accomplished by sequencing, mini sequencing, hybridization, restriction fragment analysis, oligonucleotide ligation assay or allele specific PCR.

7. An isolated polynucleotide comprising at least 10 contiguous nucleotides of SEQ ID NO: 1, or the complement thereof, and containing at least one single nucleotide polymorphism at position 216 or 563 of SEQ ID NO: 1 wherein said at least one single nucleotide polymorphism is associated with a disease, condition or disorder selected from the group consisting of breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder.

8. The isolated polynucleotide of claim 7, wherein at least one single nucleotide polymorphism is selected from the group consisting of C216->G and G563->A and the complements thereof namely G216->C and C563->T.

9. The isolated polynucleotide of claim 7, wherein said at least one single nucleotide polymorphism is located at the 3′ end of said nucleic acid sequence.

10. The isolated polynucleotide of claim 7, further comprising a detectable label.

11. The isolated nucleic acid sequence of claim 10, wherein said detectable label is selected from the group consisting of radionuclides, fluorophores or fluorochromes, peptides, enzymes, antigens, antibodies, vitamins or steroids.

12. A kit comprising at least one isolated polynucleotide of at least 10 contiguous nucleotides of SEQ ID NO: 1 or the complement thereof, and containing at least one single nucleotide polymorphism associated with a disease, condition, or disorder selected from the group consisting of breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder; and instructions for using said polynucleotide for detecting the presence or absence of said at least one single nucleotide polymorphism in said nucleic acid.

13. The kit of claim 12 wherein said at least one single nucleotide polymorphism is located at position 216 or 563 of SEQ ID NO: 1.

14. The kit of claim 13 wherein said at least one single nucleotide polymorphism is selected from the group consisting of C216->G and G563->A and the complements thereof namely G216->C and C563->T.

15. The kit of claim 12, wherein said single nucleotide polymorphism is located at the 3′ end of said polynucleotide.

16. The kit of claim 12, wherein said polynucleotide further comprises at least one detectable label.

17. The kit of claim 16, wherein said label is chosen from the group consisting of radionuclides, fluorophores or fluorochromes, peptides enzymes, antigens, antibodies, vitamins or steroids.

18. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease, condition, or disorder selected from the group consisting of breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectorny, degenerative joint disease, end stage renal disease and frequent de-clots, end stage renal disease due to focal segmental glomerular sclerosis, end stage renal disease due to insulin dependent diabetes mellitus, and seizure disorder; and instructions for using said polynucleotide for detecting the presence or absence of said single nucleotide polymorphism in a biological sample containing nucleic acid.

19. The kit of claim 18, wherein said single nucleotide polymorphism site is located at position 216 or 563 of SEQ ID NO: 1.

20. The kit of claim 19, wherein said at least one polynucleotide further comprises a detectable label.

21. The kit of claim 20, wherein said detectable label is chosen from the group consisting of radionuclides, fluorophores or fluorochromes, peptides, enzymes, antigens, antibodies, vitamins or steroids.

22. A method for treatment or prophylaxis in a subject comprising:
obtaining a sample of biological material containing nucleic acid from a subject; analyzing said nucleic acid to detect the presence or absence of at least one single nucleotide polymorphism in SEQ ID NO: 1 or the complement thereof associated with a disease, condition, or disorder selected from the group consisting of breast cancer, prostate cancer stage D, colon cancer, lung cancer, hypertension, atherosclerotic peripheral vascular disease due to hypertension, cerebrovascular accident due to hypertension, cataracts due to hypertension, hypertensive cardiomyopathy, myocardial infarction due to hypertension, end stage renal disease due to hypertension, non-insulin dependent diabetes mellitus, atherosclerotic peripheral vascular disease due to non-insulin dependent diabetes mellitus, cerebrovascular accident due to non-insulin dependent diabetes mellitus, ischemic cardiomyopathy, ischemic cardiomyopathy with non-insulin dependent diabetes mellitus, myocardial infarction due to non-insulin dependent diabetes mellitus, atrial fibrillation without valvular disease, alcohol abuse, anxiety, asthma, chronic obstructive pulmonary disease, cholecystectomy, degenerative joint disease, end stage renal disease and frequent de-clots, end stage renal disease due to focal segmental glomerular sclerosis, end stage renal disease due to insulin dependent diabetes mellitus, and seizure disorder; and treating said subject for said disease, condition or disorder.

23. The method of claim 22 wherein said nucleic acid is selected from the group consisting of DNA, cDNA, RNA and mRNA.

24. The method of claim 22, wherein said at least one single nucleotide polymorphism is located at position 216 or 563 of SEQ ID NO: 1.

25. The method of claim 22 wherein said at least one single nucleotide polymorphism is selected from the group of C216->G and G563->A and the complements thereof namely G216->C and C563->T.

26. The method of claim 22 wherein said treatment counteracts the effect of said at least one single nucleotide polymorphism detected.


SEQUENCE LISTING (6 sequences)

SEQ ID NO: 1 - DNA, 2205 bp, Homo sapiens
ggatccttagcaggggagtaacatggatttggaaagatcactttggctgctgtgtggggatagataagacggtgggagcctagaaaggaggctgggttggaaactctgggacagaaacccagagaggaaaagactgggcctggggtctccagtgagtatcagggagtggggaatcagcaggagtctggtccccacccatccctcctttcccctctctctcctttcctgcaggctggccccggctccatttccaggtgtggtcccaggacagctttggccgctgccagcttgcaggctatggattttgccatgtgcccagtagcccgggcacccaccagctggcctgccccacgtggcggcccctgggcagttggcgagaacagttggcacgggctttcgtgggtggtgggccgcagctgctgcatggggacaccatctacagtggggccgaccgctatcgcctgcacacagctgctggtggcaccgtgcacctggagatcggcctgctgctccgcaacttcgaccgctacggcgtggagtgctgagggactctgcctccaacgtcaccaccatccacaccccggacacccagtgatgggggaggatggcacagtggtcaagagcacagactctagagactgtcagagctgaccccagctaaggcatggcaccgcttctgtcctttctaggacctcggggtccctctgggcccagtttccctatctgtaaattggggacagtaaatgtatggggtcgcagggtgttgagtgacaggaggctgcttagccacatgggaggtgctcagtaaaggagagcaattcttacaggtgtctgcctcctgacccttccatccctcaggtgtcctgttgccccctcctcccactgacaccctccggaggcccccatgttgacagaccctccttctcctaccttgtttcccagcctgactctccttccgttctgggtccccctcctctggtcggctcccctgtgtctcatcccccggattaagccttctccgcctggtcctctttctctggtgacccacaccgcccgcaaagccacagcgcatctggatcacccgctttggtggcgcttggccgccaggaggcagcaccctgtttgcggggcggagccggggagcccgccccctttcccccagggctgaagggacccccctcggagcccgcccacgcgagatgaggacggtggcccagcccccccatgccctccccctgggggccgcccccgctcccgccccgtgcgcttcctgggtggggccgggggcggcttcaaaaccccctgccgacccagccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatg

SEQ ID NO: 2 - DNA, 21 bp, Homo sapiens
  misc_feature (1)..(21): Primer
cctttcccctctctctccttt

SEQ ID NO: 3 - DNA, 19 bp, Homo sapiens
  misc_feature (1)..(19): Primer
gatggtggtgacgttggag

SEQ ID NO: 4 - DNA, 19 bp, Homo sapiens
  misc_feature (1)..(19): Primer
atggggacaccatctacag

SEQ ID NO: 5 - DNA, 21 bp, Homo sapiens
  misc_feature (1)..(21): Primer
tcttgaccactgtgccatcct

SEQ ID NO: 6 - DNA, 14 bp, Homo sapiens
  misc_feature (11)..(11): n=any nucleotide
ccyytyyytyntty